(19) United States
(12) Patent Application Publication
(10) Pub. No.: US 2012/0245559 A1
(43) Pub. Date: Sep. 27, 2012

(54) Grip for self-injectable device

(76) Inventor: Stanley Kruger, Albuquerque, NM (US)

(21) Appl. No.: 13/506,073

(22) Filed: Mar. 23, 2012

Related U.S. Application Data
(60) Provisional application No. 61/467,490, filed on Mar. 25, 2011.

(51) Int. Cl. A61M 5/31

(52) U.S. Cl. 604/506; 604/207

(57) ABSTRACT

The present invention, in one embodiment, is a gripping apparatus to ergonomically grip a self-injectable device (such as, for example, an insulin pen) to thereby provide a user's fingers with a gripping means on the self-injectable device so that the combination of the apparatus with the self-injectable device facilitates one-handed operation on various parts of the human body and comprises a complete operational self-injectable assembly. The apparatus is adapted to snap-fit the body of a self-injectable device (e.g., an insulin pen) by inserting the self-injectable device longitudinally through the flange apertures of the apparatus, and twisting the apparatus around the device's body until the apparatus is in snug engagement with the device. The apparatus assists the user in using the self-injecting device for injection on any portion of the human body.

### PRIORITY CLAIM

The present invention claims priority from U.S. Provisional Patent Application No. 61/467,490, filed Mar. 25, 2011 and entitled “Grip For Self-Injectable Device”.

### FIELD OF INVENTION

The present invention relates generally to medication dispensing devices, and more particularly, to a gripping means for a self-injectable device, such as, for example and without limitation, an insulin pen.

### BACKGROUND OF THE INVENTION

By way of background information, insulin pens are generally described as a self-injecting device which measures insulin and allows a user to inject the insulin into the user's body at a predetermined location. As seen generally in FIG. 1 and the remaining drawings, typical insulin pens 120 resemble a writing pen with a cap, having an elongated body which holds the insulin cartridge. At one end of each pen is a needle retainer portion 121, adapted to removeably store injecting needles 129. Adjacent to the needle retainer portion 121 is typically found the cartridge storage portion 123 which is covered by a cover or cap (not shown, but well known in the art). The internal cartridge storage portion may take up substantially the length of the cap, or the cartridge may proceed further into a dosage control portion 125 on the device. In most instances, the circumferential surface width of the cartridge storage portion 123 is smaller than the circumferential surface width of the dosage control portion 125 of the device 120 (see e.g., FIG. 7) so that the area joining the cartridge storage portion and the dosage control portion is defined by a circumferential ledge L. In this regard, when a cap or cover is inserted onto the device, the entire longitudinal circumferential width of the outer housing of the device is substantially the same (similar to a conventional pen used for writing). At the opposite end of each device is a plunger or button 127, which is adapted to dispense insulin from the cartridge into and through the needle 129 when the button 127 is pressed or otherwise engaged by a user. The pen's elongated body usually further contains at least one tab Tₓ (where x=1, 2, 3, etc.), wherein each tab is normally used to engage and maintain the pen's cap on the pen when the pen is not in use.

Insulin pens are usually used to treat blood sugar irregularities associated with diabetes. Some insulin pens contain insulin cartridges that are replaceable, while other pens require a new pen every time the insulin is empty. Insulin pens typically operate with disposable needles 121 that are replaced after each use.

Most insulin pens work in the same way for the use, namely: 1) removing the cap on the insulin pen; 2) determine if there is enough insulin remaining in the cartridge for injection through the pen's typically translucent or transparent level indicators; 3) apply the skin area the user will inject the insulin into their body with a sterile alcohol swab; 4) screw or snap the disposable needle onto the insulin pen, then remove the packaging and both covers from the needle; 5) prime the insulin pen to remove any air bubbles; 6) pointing the insulin pen upward, so the needle is facing toward the ceiling, and turning the dial on the insulin pen to dispense Y units of insulin (the value “Y” being dependant on each user), then pressing the plunger or button at the tip opposite the needle of the pen, while watching the needle to make sure a small amount of insulin comes out of the need; and 7) setting a dose on the pen dependent on each user (usually based on the type of food eaten or the user's current measured blood sugar level; 8) holding the end of the insulin pen in the first with the thumb or palm ready to depress the plunger (e.g., FIG. 1), inserting the needle into the skin and press the pen's plunger or button down with your thumb until the dose reads zero (many pens may also make a clicking noise during this process); and 9) leaving the needle inserted into the skin for approximately ten seconds for insulin injection into the body, removing the needle and cleaning the skin area if needed with a sterile cotton ball swap or like sterile material.

A major problem exists with the use of insulin pens, especially for those users of insulin pens who have arthritis or cannot properly and steadily hold insulin pens during injection for a number of reasons. First, injecting insulin from a pen requires a steady hand. If a user applies pressure to the pen's button at an angle not approximating ninety degrees, the user's thumb will likely slip off of the button without injection. Moreover, insulin pen users usually have to inject insulin multiple times a day, so that selecting a body part that has not already been subjected to needle sticks (and thus, being sore) is difficult. This means that a user will then attempt to inject insulin in body portions that normally would not be used (such as the front abdomen, upper legs, bicep areas, lower back areas, buttocks, and back legs areas and the like as generally show in FIG. 10), but attempting to hold the pen for injection in these body areas is difficult. The known prior art does not discuss these problems. The present invention solves these problems.

U.S. Pat. No. 6,454,746 B1 to Bydlon et al. discloses a medication delivery apparatus which is generally defined as an insulin pen having an integrally formed, single contoured grip on the pen. While this disclosure discusses many of the problems associated with using an insulin pen, it too, is deficient in the art. Namely, the apparatus in this invention requires a user to hold the apparatus in an unnatural position (e.g. in a hand position similar to holding a knife during a stabbing movement), and is therefore difficult to use except for those portions of the body that are readily accessible (e.g., generally, the front portion of the human body).

Accordingly, there is a need for a novel gripping means for self-injectable devices which permit easy and convenient handling of the self-injectable device. There is also a need for a novel gripping means for use with varying sizes and lengths of self-injectable devices. A need also exists for a gripping means adapted to support a self-injectable device in fixed relationship (even after repeated uses), and one which is inexpensive to manufacture. A need further exists for a novel gripping means for a self-injectable device which is oriented to account for the natural angular relationship between a user's fingers and the user's body portions when using the self-injectable device.

It is therefore an exemplary object of the present invention to provide a gripping means for use with a self-injectable device which preferably may be used more than once.

It is also an exemplary object of the present invention to provide a gripping means for use with a self-injectable device which is preferably disposable.

It is also an exemplary object of the present invention to provide a gripping means for use with a self-injectable device permitting easy, one-handed use, thereby promoting efficient use of the self-injectable device.

It is also an exemplary object of the present invention to provide a gripping means for use with a self-injectable device which is inexpensive to manufacture and simple to use.

Other objects and benefits of the present invention will now be recognized by those of skill in the art.

### SUMMARY OF THE INVENTION

The following summary of the invention is provided to facilitate an understanding of some of the innovative features unique to the present invention, and is not intended to be a full description of variations that may be apparent to those of skill in the art. A full appreciation of the various aspects of the invention can be gained from the entire specification, claims, drawings, and abstract taken as a whole.

The present invention, in one embodiment, is a gripping apparatus adapted to ergonomically grip a self-injectable device (such as, for example, an insulin pen) to thereby provide a user's fingers with a gripping means on the self-injectable device so that the combination of the apparatus with the self-injectable device facilitates one-handed operation on various parts of the human body and comprises a complete operational self-injectable assembly. The apparatus is adapted to snap-fit the body of a self-injectable device (e.g., an insulin pen) by inserting the self-injectable device longitudinally through the flange apertures of the apparatus, and twisting the apparatus approximately between 5 and 90 degrees around the device's body (the angular displacement being dependant on the type of self-injectable device used) until the apparatus is in snug engagement with the device (which usually is either felt or occurs with a “snap” or “click” sound). The apparatus assists the user in using the self-injecting device on any portion of the human body.

The novel features of the present invention will become apparent to those of skill in the art upon examination of the following detailed description of the preferred embodiment or can be learned by practice of the present invention. It should be understood, however, that the detailed description of the preferred embodiment and the specific examples presented, while indicating certain embodiments of the present invention, are provided for illustration purposes only because various changes and modifications within the spirit and scope of the invention will become apparent to those of skill in the art from the detailed description, drawings and claims that follow.

### BRIEF DESCRIPTION OF THE DRAWINGS

The accompanying figures further illustrate the present invention and, together with the detailed description of the preferred embodiment, assists to explain the general principles according to the present invention.

FIG. 1 illustrates a side perspective view of one exemplary embodiment of the present invention using a person's primary index finger to operate the self-injectable device, the dashed line structure representing an exemplary self-injectable device;

FIG. 2 is a top side view of the embodiment illustrated in FIG. 1;

FIG. 3 is a bottom side view of the embodiment illustrated in FIG. 1;

FIG. 4 is a side view of the embodiment illustrated in FIG. 1;

FIG. 5 is a ninety degree (90°) rotated side view (right to left rotation, which is the mirror image of the view from left to right rotation) of the embodiment illustrated in FIG. 4;

FIG. 6a is a side perspective view of the embodiment illustrated in FIG.

1;

FIG. 6b is a one hundred and eighty degree (180°) from top to bottom rotated side perspective view of the embodiment illustrated in FIG. 6a;

FIG. 7 illustrates the embodiment shown in FIG. 1 as being insertable into a self-injectable device;

FIG. 8 illustrates the embodiment shown in FIG. 7 as being insertable into a self-injectable device so that the device's tabs substantially register with and pass through the embodiment's notches, wherein the apparatus is rotated about the device's body;

FIG. 9 illustrates the embodiment of FIG. 8 as being removable from the self-injectable device after the embodiment is rotated in the opposite direction shown in FIG. 8;

FIG. 10 illustrates exemplary locations where a self-injectable device may be used on the human body, this illustration depicting locations which may be difficult to use a self-injectable device; and

FIG. 11 illustrates an optional use of the present invention, wherein complimentary needles and alcohol swaps are stored within the body of the present invention.

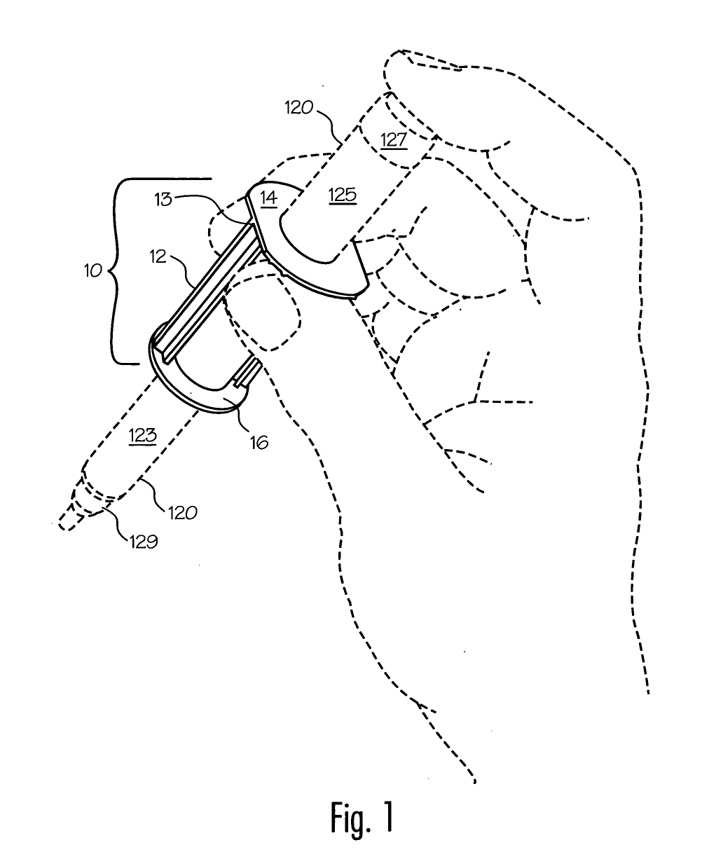

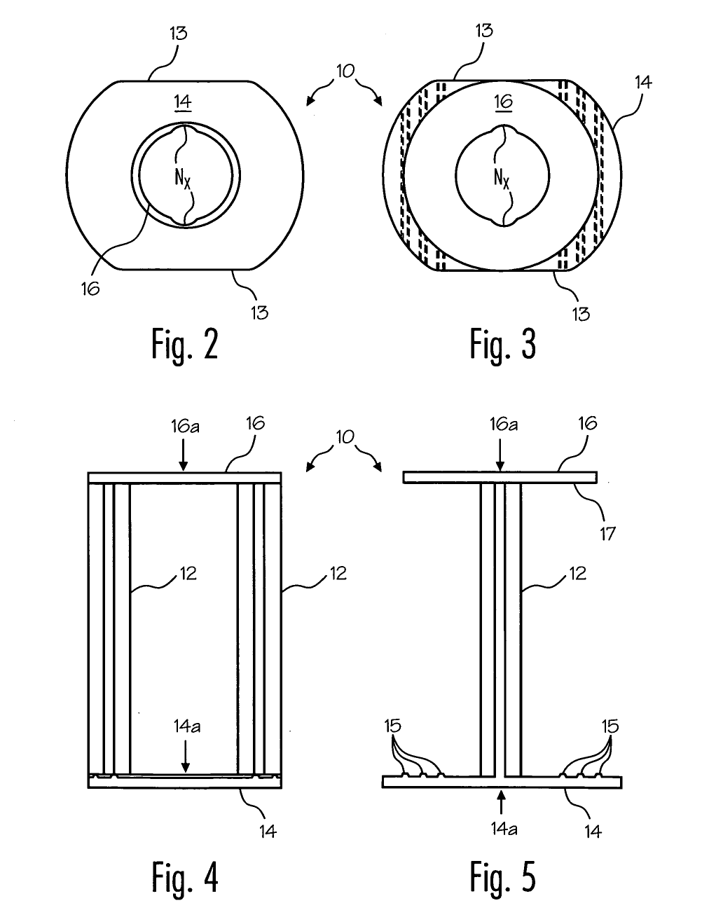

Additional aspects of the present invention will become evident upon reviewing the non-limiting embodiments described in the specification and the claims taken in conjunction with the accompanying figures, wherein like reference numerals denote like elements.

Upon review of the present disclosure, those of skill in the art will realize that the present invention may be embodied as a system, kit, assembly, process, method or apparatus. Other variations and modifications of the present invention will be apparent to those of ordinary skill in the art, and is not limited except by the appended claims. The particular designs and configurations discussed herein can be varied, and are cited to illustrate particular embodiments of the present invention. It is contemplated that the use of the present invention can involve components having different characteristics as long as the principles disclosed herein are followed.

### DETAILED DESCRIPTION OF THE PREFERRED EMBODIMENT

The present invention resides in a novel gripping apparatus for use in snug engagement with a self-injectable device, whether separately, as a method or process, or as a kit, the apparatus being used to hold the self-injectable device in a comfortable but controllable position during the injection process.

Embodiments of the present invention comprise a grip or gripping means that is used for self-injectable devices, such as, for example, an insulin pen. The grip preferably allows a user, particularly users with arthritis, to inject themselves in many areas of the body using only one hand. The grip allows a user to inject using the left or right hand without the user's fingers slipping off or away from the self-injectable device.

In one embodiment, the present invention is a gripping means 10 comprising a flange head 14 at a first end, a flange tail 16 at a second end, and at least one support structure 12 coupling the flange head 14 with the flange tail 16. Flange head 14 has a bore or an aperture 14a formed therethrough in substantially a center portion of the head 14. Aperture 14a has a circumferential width which is substantially the same or slightly larger than the circumferential width of dosage control portion 125, so that flange head 14 can engage dosage control portion 125 in snug relationship. Flange tail 16 has a bore or an aperture 16a formed therethrough in substantially a center portion of the tail 16. Aperture 16a has a circumferential width which is substantially the same or slightly larger than the circumferential width of cartridge storage portion 123, so that flange tail 16 can engage cartridge storage portion 123 in snug relationship.

The planar shape of flange head 14, in one embodiment, is oval or ellipsoid shaped. The planar shape of flange tail, preferably, may be substantially circular. In another embodiment as seen in the figures, the flange head 14 is preferably oval or ellipsoid shaped with the longer axis of symmetry 13 being substantially flat, which when in combined engagement with a self-injectable device 120, prevents the overall gripping means/device from rolling across a flat surface (such as a desk or table, for example). Of course, those of skill in the art will recognize that the exact planar shape of either the flange head 14 or the flange tail 16 may be made of any known geometric shape for any other beneficial reason.

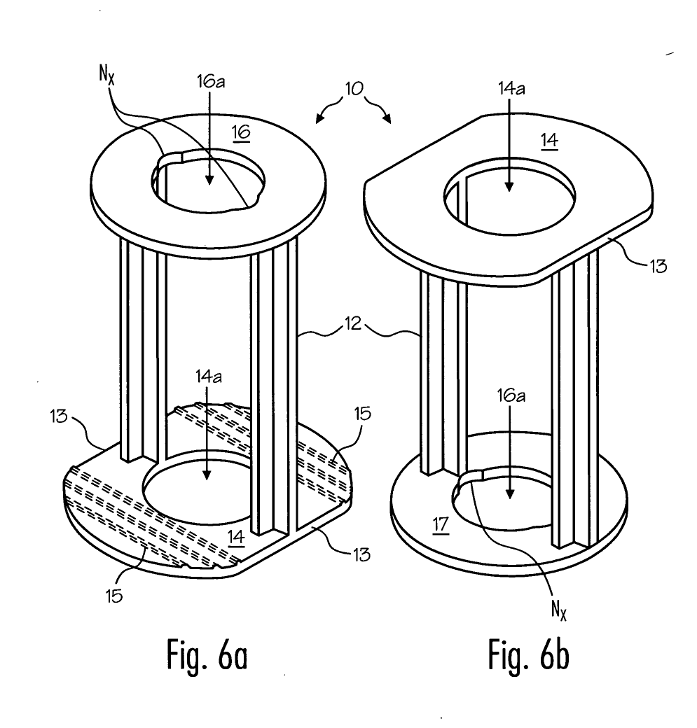

Preferably, support structure 12 is an elongated structure which mechanically communicates with flange head 14 and flange tail 16. While the preferred embodiment of support structure 12 discloses two column structures (see e.g., FIGS. 6a, 6b) which are generally of cross-hatch (or, x-shaped) construction which are believed to be more rigid than a typical tubular column, those of skill in the art will recognize that other geometrical designs are easily available for use with the present invention as long as the support structure provides support between the flange head 14 and the flange tail 16 during operation so that the apparatus does not break.

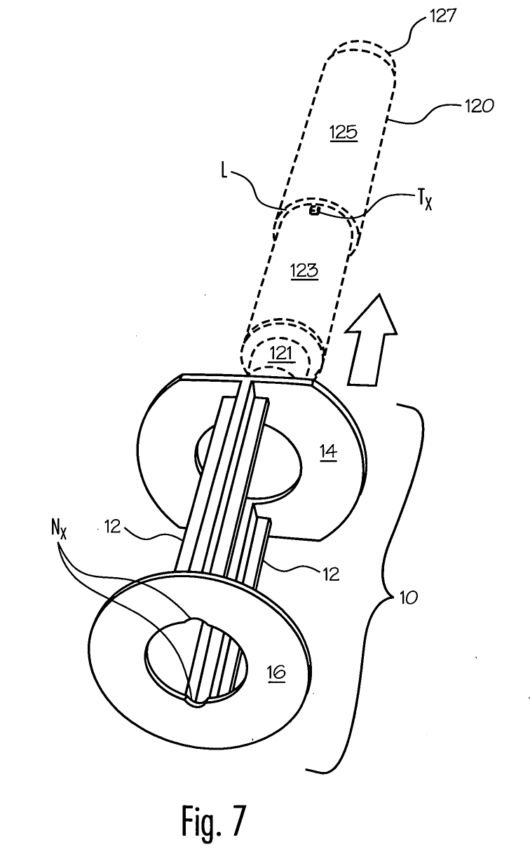

One key feature of the present invention is the formation of one or more notches Nₓ (where x=1, 2, 3, etc.) in the flange tail's aperture 16a. As described previously, typical self-injectable devices such as an insulin pen have one or more tabs Tₓ formed on the pen's outer-housing (see e.g., FIG. 7) which are used to hold or retain a cap or cover for the device. The present invention capitalizes on these conventional tabs by adapting each notch Nₓ to receive any tab Tₓ formed on the device in passing (insertable or removable) relationship so that when the present invention is inserted onto or removable from a self-injectable device.

In one optional embodiment, the flange head has a lower surface 15 which faces the flange tail 16. Preferably, as seen for example in FIGS. 3 and 6a, at least one or more friction means (such as, for example, one or more ridges which raise from surface 15) may be formed or placed on the flange head lower surface 15 to provide additional friction for a user's fingers when the apparatus is in operation.

The present invention therefore, in one embodiment, is a gripping means or gripping apparatus 10 adapted to grip a self-injectable device (such as, for example, an insulin pen) to thereby provide a user's fingers with a gripping means on the self-injectable device so that the combination of the apparatus with the self-injectable device comprise a complete operational self-injectable assembly. The apparatus 10 is adapted to snap-fit the body of a self-injectable device (e.g., 120) by inserting the self-injectable device longitudinally through the flanges of apparatus 10, and twisting the apparatus 10 approximately between 5 and 180 degrees around the device's body (the angular displacement being dependant on the type of self-injectable device used) until the apparatus 10 is in snug engagement with the device.

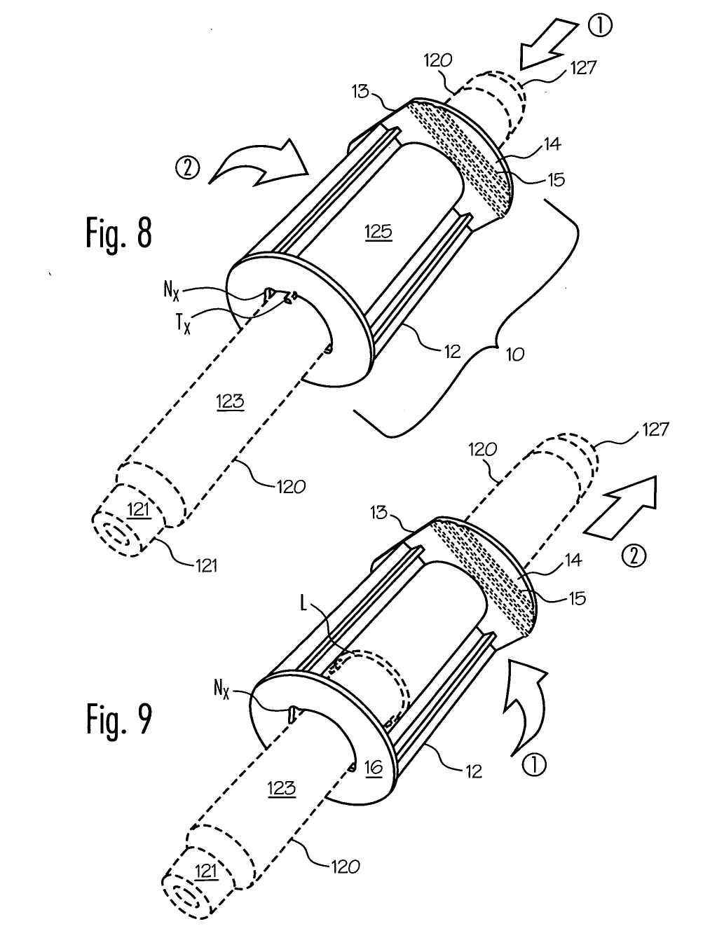

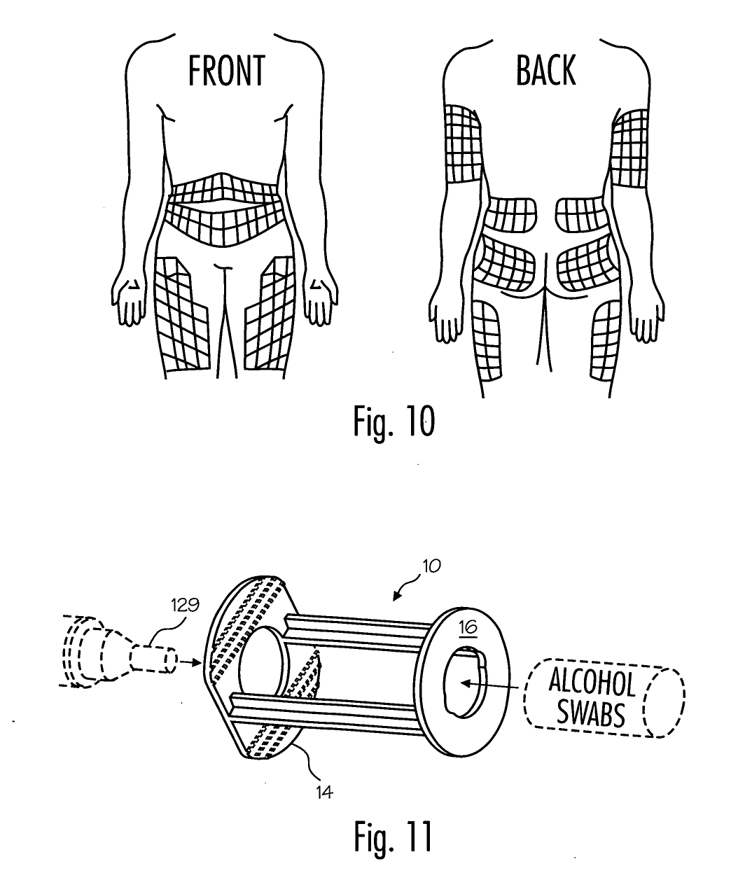

In operation as shown generally in FIGS. 8 and 9, the needle end portion 121 (adjacent to where a needle assembly 129 connects to the device) of self-injectable device 120 will be first inserted through the flange head aperture 14a and then through the flange tail aperture 16a, so that each tab Tₓ (where x=1, 2, 3, etc.) on the self-injectable device 120 registers or otherwise lines up with each notch Nₓ (where x=1, 2, 3, etc.) on the flange tail 16, and passes through each notch N_{X} in insertable passing relationship (shown generally as arrow 1 in FIG. 8). The apparatus 10 may then be rotated about the device 120 from approximately 5 to 90 degrees (shown generally as arrow 2 in FIG. 8), depending on the construction of the self-injectable device 120. In most case, a physical “snap” is felt or heard when the apparatus 10 achieves substantial snug or engagement relationship with the device 120, and the device 120 is thereafter ready for injection into a preselected portion of the human body (such as, for example but not by limitation, those areas depicted in FIG. 11). For injection, a user will engage the combined apparatus 10/device 120 (thereby defining a medication dispensing device) and grasp the lower surface 15 of flange head 14 with the thumb and middle finger while placing the index finger (or, forefinger) on the button 127, place the device's needle assembly 129 on the area of the human body where injection is to take place, and allow the user to press the button 127 for injection. In this regard, a user will use the fingers to place a longitudinal force to the flange head lower surface 15 in order to engage the button 127, the longitudinal force being counterbalanced by the force through the support structure 12 which is put on the flange tail 16 as it engages the circumferential ledge L. As a consequence, the needle assembly 129 may be placed against the human body while the combined gripping means/device is held firmly allowing the device's button 127 to be pressed for injection.

To remove the apparatus 10 from the device 120, the apparatus 10 may then be rotated about the device 120 in a reverse direction (or, counter-rotated) shown in FIG. 8 (shown generally as arrow 1 in FIG. 9), and slideably remove the apparatus 10 by having each tab Tₓ pass through each notch Nₓ in removable passing relationship (shown generally as arrow 2 in FIG. 9).

Those of skill in the art will now recognize that the gripping means 10 may be provided as a stand-alone product, it may be used in combination with a self-injectable device as part of a kit, or it may be used as part of a method. Preferably, the gripping means 10 may be manufactured from a plastic material which has the desired characteristics, such as easy to mold, inexpensive and having strong shape retention qualities after repeated uses. If desired, the present apparatus 10 may also be integrally formed with a self-injectable device.

Optionally, as seen in FIG. 11, aperture 16a of the flange tail 16 may be adapted to removably store a device's needle assembly. Further, aperture 14a of the flange head 14 may adapted to removably store additional medical objects such as an alcohol swab package.

It should, be appreciated that the particular implementations disclosed herein are illustrative of the invention and its best mode, and are not intended to otherwise limit the scope of the present invention in any way. Benefits, other advantages, and solutions to problems have been described above with regard to specific embodiments. However, the benefits, advantages, solutions to problems, and any element(s) that may cause any benefit, advantage, or solution to occur or become more pronounced are not to be construed as critical, required, or essential features or elements of any or all the claims. As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus. Further, no element described herein is required for the practice of the invention unless expressly described as “essential” or “ critical”.

Benefits, other advantages, and solutions to problems have been described above with regard to specific embodiments. However, the benefits, advantages, solutions to problems, and any element(s) that may cause any benefit, advantage, or solution to occur or become more pronounced are not to be construed as critical, required, or essential features or elements of any or all the claims. As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus. Further, no element described herein is required for the practice of the invention unless expressly described as “essential” or “ critical”.

Those of skill in the art will realize that the present invention may be incorporated into other types of injection devices known in the art, such as those employed in the administration of needleless or intramuscular injections.

Other variations and modifications of the present invention will be apparent to those of ordinary skill in the art, and it is the intent of the appended claims that such variations and modifications be covered. The particular values and configurations discussed above can be varied, are cited to illustrate representative embodiments of the present invention and are not intended to limit the scope of the invention. It is contemplated that the use of the present invention can involve components having different characteristics as long as the principle is followed.

I claim:

1. A gripping apparatus for a self-injectable device which is defined by a needle retainer portion, a dosage control portion, and a cartridge storage portion therebetween, the cartridge storage portion having at least one tab formed thereon, the cartridge storage portion having a smaller circumferential surface width than the circumferential surface width of the dosage control portion so that the area joining the cartridge storage portion and the dosage control portion is defined by a circumferential ledge, the gripping apparatus comprising:
a flange head having a flange head aperture formed therethrough;
a flange tail having a flange tail aperture formed therethrough; and
at least one support structure coupling the flange head and the flange tail,
the circumferential width of flange head aperture being substantially the same as the circumferential width of the dosage control portion so that flange tail can engage cartridge storage portion in snug relationship, the circumferential width of flange tail aperture being substantially the same as the circumferential width of the cartridge storage portion, the flange tail further having at least one notch formed adjacent to the flange tail aperture, each notch on the apparatus adapted to receive any tab in insertable or removably passing relationship with the device.

2. The gripping apparatus of claim 1 wherein the flange head has a ellipsoid planar shape.

3. The gripping apparatus of claim 1 wherein the flange head has a planar ellipsoid shape having a longer axis of symmetry being substantially flat.

4. The gripping apparatus of claim 1 wherein at least one support structure is formed as a cross hatch.

5. The gripping apparatus of claim 1 wherein the flange head has a lower surface facing the flange tail, the lower surface having a friction means thereon.

6. The gripping apparatus of claim 1 wherein the flange head has a planar ellipsoid shape having a longer axis of symmetry being substantially flat, and at least one support structure is formed as a cross hatch.

7. The gripping apparatus of claim 6 wherein the flange head has a lower surface facing the flange tail, the lower surface having a friction means thereon.

8. The gripping apparatus of claim 1, the flange head being adapted to removably store a device's needle assembly, the flange tail being adapted to store one or more medical objects.

9. A kit for dispensing medicine, the kit comprising:
a self-injectable device which is defined by a needle retainer portion, a dosage control portion, and a cartridge storage portion therebetween, the cartridge storage portion having at least one tab formed thereon, the cartridge storage portion having a smaller circumferential surface width than the circumferential surface width of the dosage control portion so that the area joining the cartridge storage portion and the dosage control portion is defined by a circumferential ledge; and
a gripping apparatus comprising a flange head having a flange head aperture formed therethrough, a flange tail having a flange tail aperture formed therethrough, and at least one support structure coupling the flange head and the flange tail, the flange head aperture being substantially the same circumferential width as the dosage control portion so that flange tail can engage cartridge storage portion in snug relationship, the flange tail aperture being substantially the same circumferential width as the cartridge storage portion, the flange tail further having at least one notch formed adjacent to the flange tail aperture, each notch on the apparatus adapted to receive any tab in insertable or removably passing relationship with the self-injectable device.

10. The kit of claim 9 wherein the flange head has a ellipsoid planar shape.

11. The gripping apparatus of claim 9 wherein the flange head has a planar ellipsoid shape having a longer axis of symmetry being substantially flat.

12. The gripping apparatus of claim 9 wherein at least one support structure is formed as a cross hatch.

13. The gripping apparatus of claim 9 wherein the flange head has a lower surface facing the flange tail, the lower surface having a friction means thereon.

14. The gripping apparatus of claim 9 wherein the flange head has a planar ellipsoid shape having a longer axis of symmetry being substantially flat, and at least one support structure is formed as a cross hatch.

15. The gripping apparatus of claim 14 wherein the flange head has a lower surface facing the flange tail, the lower surface having a friction means thereon.

16. A method of dispensing medication from a self-injectable device using a gripping means, the steps comprising:
obtaining the self-injectable device, the device being defined by a needle retainer portion adapted to retain a removable needle assembly, a dosage control portion, a cartridge storage portion therebetween, and a button adjacent to the dosage control portion, the cartridge storage portion having at least one tab formed thereon, the cartridge storage portion having a smaller circumferential surface width than the circumferential surface width of the dosage control portion so that the area joining the cartridge storage portion and the dosage control portion is defined by a circumferential ledge, the button adapted to dispense medication through a needle assembly on the device when the button is engaged;
introducing a gripping means comprising a flange head having a flange head aperture formed therethrough, a flange tail having a flange tail aperture formed therethrough, and at least one support structure coupling the flange head and the flange tail, the flange head aperture being substantially the same circumferential width as the dosage control portion so that flange tail can engage cartridge storage portion in snug relationship, the flange head further having a lower surface facing the flange tail, the flange tail aperture being substantially the same circumferential width as the cartridge storage portion, the flange tail further having at least one notch formed adjacent to the flange tail aperture, each notch on the apparatus adapted to receive any tab in insertable or removably passing relationship with the self-injectable device;
inserting the device's needle assembly through the flange head aperture and through the flange tail aperture so that each tab registers with and passes through each notch in insertable passing relationship until the flange tail contacts the circumferential ledge;
rotating the gripping means about the device until the gripping means achieves substantial engagement with the device to thereby define a medication dispensing device; and
directing a user to grasp the flange head lower surface with the user's fingers, placing the device on the user's body where injection is required, and engaging the device's button for injection.

17. The method of claim 16 wherein the step of rotating the gripping means about the device further comprises the step of rotating the gripping means about the device from approximately 5 to 90 degrees.

18. The method of claim 16 further comprising the step of counter-rotating the gripping means about the device until each tab registers with each notch, and slideably removing the gripping means from the device by having each tab pass through each notch.

19. The method of claim 16 wherein the step of directing a user to grasp the flange head lower surface with the user's fingers, placing the device on the user's body where injection is required, and engaging the device's button for injection further comprises the step of allowing the user to place a longitudinal force to the flange head lower surface in order to engage the button, the longitudinal force being counterbalanced by the force through the support structure which is put on the flange tail as it engages the circumferential ledge.

20. The method of claim 16 wherein the flange head has a planar ellipsoid shape having a longer axis of symmetry being substantially flat.
